SEQUENCE LISTING (11 sequences)

SEQ ID NO: 1 - DNA, 28 bp, Artificial
  chemically synthesized
caattctgcagttgcgttcacgtcgttg

SEQ ID NO: 2 - DNA, 28 bp, Artificial
  chemically synthesized
taaggctcatgaaaaacacagctatcgc

SEQ ID NO: 3 - DNA, 378 bp, Escherichia coli
  ompA-signal peptide (1)..(63)
  RNase T1 wildtype (64)..(378)
atgaaaaacacagctatcgcgattgcagtggcactggctggtttcgctaccgtagcgcaggccgcatgcgactacacttgcggttctaactgctactcttcttcagacgtttctactgctcaggcggccggatataaacttcacgaagacggtgaaactgttggatccaattcttacccacacaagtacaacaactacgaaggttttgatttctctgtgagctctccctactacgaatggcctatcctctcgagcggtgatgtttactctggtgggtccccgggtgctgaccgtgtcgtcttcaacgaaaacaaccaactagctggtgttatcactcacactggtgcttctggtaacaacttcgttgaatgtacataa

SEQ ID NO: 4 - DNA, 378 bp, Escherichia coli
  ompA-signal peptide (1)..(63)
  RNaseT1-His92Ala (64)..(378)
atgaaaaacacagctatcgcgattgcagtggcactggctggtttcgctaccgtagcgcaggccgcatgcgactacacttgcggttctaactgctactcttcttcagacgtttctactgctcaggcggccggatataaacttcacgaagacggtgaaactgttggatccaattcttacccacacaagtacaacaactacgaaggttttgatttctctgtgagctctccctactacgaatggcctatcctctcgagcggtgatgtttactctggtgggtccccgggtgctgaccgtgtcgtcttcaacgaaaacaaccaactagctggtgttatcactgccactggtgcttctggtaacaacttcgttgaatgtacataa

SEQ ID NO: 5 - DNA, 7336 bp, Artificial
  Plasmid pA2T1
taggcgtatcacgaggccctttggataaccagaagcaataaaaaatcaaatcggatttcactatataatctcactttatctaagatgaatccgatggaagcatcctgttttctctcaatttttttatctaaaacccagcgttcgatgcttctttgagcgaacgatcaaaaataagtgccttcccatcaaaaaaatattctcaacataaaaaactttgtgtaatacttgtaacgctacatggagattaactcaatctagctagagaggctttacactttatgcttccggctcgtataatgtgtggaattgtgagcggataacaatttcacacaggaaacagctatgaccatgattacggattcactggaactctagataacgaggcgcaaaaaatgaaaaacacagctatcgcgattgcagtggcactggctggtttcgctaccgtagcgcaggccgcatgcgactacacttgtggttccaactgctactcttcttcagacgtttctactgctcaagcggccggatataaacttcacgaagacggtgaaactgttggatccaattcttacccacacaaatacaacaactacgaaggttttgatttctctgtgagctctccctactacgaatggcctatcctctcgagcggtgatgtttactctggtgggtccccgggtgctgaccgtgtcgtcttcaacgaaaacaaccaactagctggtgttatcactcacactggtgcttctggtaacaacttcgttgaatgtacataagcttggatcgatccgggctgagcaacgacgtgaacgcaatgcgttccgacgttcaggctgctaaagatgacgcagctcgtgctaaccagcgtctggacaacatggctactaaataccgcaagtaatagtacctgtgaagtgaaaaatggcgcacattgtgcgacattttttttgtctgccgtttaccgctactgcgtcacgcgtaacatattcccttgctctggttcaccattctgcgctgactctactgaaggcgcattgctggctgcgggagttgctccactgctcaccgaaaccggataccctgcccgacgatacaacgctttatcgactaacttctgatctacagccttattgtctttaaattgcgtaaagcctgctggcagtgtgtatggcattgtctgaacgttctgctgttctcctgccgatagtggtcgatgtacttcaacataacgcatcccgttaggctccacggaatatttcaccggttcgttgatcactttcaccggcgttcccgtccgcacgctggagaacaaggctttaatatccggtgcattcatgcgaatacaccctgaactgacgcgcaaaccgacgctgtccggcgcactggtaccatgaatgaggtattcgccattaccatgcgcgaggcgcagtgcgtaacgtcctagcgggttatttggtccggcaggaacgactggcggtaatttaatgccacgctccagcgaacgctgacgaatgcctgccgtaggcgtccaggttgggttagggattttctgcccaacacgcgtttccatcaccggcgtttccagcccctgcaatccaatacctattggataaacctgcacaatattttctcccggcggataataataaaggcgcagctctgcaaggttgacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgctaacgataatcccctgacgcggtgcatcaggtaataacagttgtgaaggaatagttatcgtcgtaccaggttttggcaccggggcgatagtgttattggcttcaaggatcaacattgccgcagtatcaaaacgtcgggcaatagcctgaaggtttttatccccttcttgcaccgtatacgtttgattttgcccaaccagtcggcttccggttggtggtagcggataatcaaccgcccaggcagcctggatggcgctaaaagcgccgataagcgtgagtgtaagcaaagacgcgcgtttcattgtaaacctcctgtatttgccggagactcacgctgaaacgtcggatggcgcttatgttcacctgaaaccaaaacactcctgtgcaggtcagtgtaaacattgaccatccggcaatgtgagccaaccggatgaaagctgtccttttagtttagctaagtgcagcggctttggcgcgaattgcgcgaatcatcgcttccagaccttgtgaacgagatggggtgagatgttgggtgagcgccattttttcaaaccacggacgcacatcgaaattgacaatatcctgcggcgtcatctgatcgtagagaataaagacgaccgcaataagccctttcacaatcgccgcatcgctgtcgccctgtaattcaataattccctgggcattctggcgcatgacaatccacacctgactctgacagccctgaatgctattttgtggacttctgtcttcgtcgcgtaattctggcagacgctgggggaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaa

SEQ ID NO: 6 - DNA, 3653 bp, Artificial
  Plasmid pETBlue-2
taatacgactcactataggggaattgtgagcggataacaattcccctctagacttacaatttccattcgccattcaggctgcgcaactgttgggaagggcgatcggtacgggcctcttcgctattacgccagcttgcgaacggtgggtgcgctgcaaggcgattaagttgggtaacgccaggattctcccagtcacgacgttgtaaaacgacggccagcgagagatcttgattggctagcagaataattttgtttaactttaagaaggagatataccatggcgatatcccgggagctcgtggatccgaattctgtacaggcgcgcctgcaggacgtcgacggtaccatcgatacgcgttcgaagcttgcggccgcacagctgtatacacgtgcaagccagccagaactcgctcctgaagacccagaggatctcgagcaccaccaccaccaccactaatgttaattaagttgggcgttgtaatcatagtcataatcaatactcctgactgcgttagcaatttaactgtgataaactaccgcattaaagctattcgatgataagctgtcaaacatgataattcttgaagacgaaagggcctaggctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgaccggtgaattgtgagcgctcacaattctcgtgacatcataacgtcccgcgaaat

SEQ ID NO: 7 - DNA, 18 bp, Artificial
  chemically synthesized
gtggctggctggtatgga

SEQ ID NO: 8 - DNA, 18 bp, Artificial
  chemically synthesized
tatctgtgcttcggtggc

SEQ ID NO: 9 - DNA, 98 bp, Artificial
  chemically synthesized
gtaggatccaattcttacccacacnnbnnbnnbnnbnnbnnbnnbnnbnnbnnbnnbnnbnnbnnbnnbnnbnnbgaatggcctatcctctcgagcgg

SEQ ID NO: 10 - DNA, 41 bp, Artificial
  substrate “Sub_G”
ccataccagccagccacaagcaagccaccgaagcacagata

SEQ ID NO: 11 - DNA, 41 bp, Artificial
  substrate “Sub_A”
ccataccagccagccacaagcaaaccaccgaagcacagata

(19) United States
(12) Patent Application Publication
(10) Pub. No.: US 2008/0220518 A1
(43) Pub. Date: Sep. 11, 2008

(54) Method From the Selection of Biomolecules From Biomolecules Variant Libraries

(76) Inventors: Thomas Greiner-Stoffele, Leipzig (DE); Marc Struhalla, Leipzig (DE)

Correspondence Address: CROWELL & MORING LLP;INTELLECTUAL PROPERTY GROUP, P.O. BOX 14300, WASHINGTON, DC, 20044-4300, US

(21) Appl. No.: 10/576,684

(22) PCT Filed: Oct. 22, 2004
(86) PCT No.: PCT/DE04/02386

§ 371 Date: Feb. 6, 2007

(30) Foreign Application Priority Data
Oct. 23, 2003 (DE) 103 50 474.5

(51) Int. Cl. C12N 15/00; C40B 20/00; C40B 20/08

(52) U.S. Cl. 435/320.1; 506/2; 506/6

(57) ABSTRACT

The invention relates to a method from the selection of biomolecules from variant libraries, in particular of biocatalytically active biomolecules, comprising the steps: a) production of a variant library, b) division of the library into a number of compartments, which is smaller than the total number of variants in the variant library by a factor of at least 10, c) production and testing of the biomolecules in the individual compartments for a particular property, for example, a biocatalytic activity, d) selection of at least one compartment I in which there are biomolecules fulfilling the desired property, e) division of the partial library contained in the selected compartment into further compartments and f) n-fold repetition of the steps c) to e) until each compartment contains only one variant of the gene sequence coding for the biomolecule. In contrast to established methods which comprise mutagenesis and selection steps, said method starts with a large library in which the desired variant is contained from the outset.

The invention concerns a method for the selection of biomolecules from biomolecule variant libraries, in particular of enzymes or other biocatalytically active biomolecules. Biomolecules find manifold use in the technical or medicinal applications and processes. Many of the therefore needed properties of biomolecules are not present in nature or could not yet be identified. The generation of such new properties from existing biomolecules demands the production of very large variant libraries with stochastically changed compositions by the introduction of mutations. The identification of variants with the desired properties needs suitable selection- or screening-methods.

The stochastically introduction of mutations into the genetic material is also the incitement of natural evolution. Natural systems replicate with mutation rates, which lay curtly under the so called error threshold. The error threshold is the maximal mutation rate, which just not leads to an extinction of the population. With mutation rates below the error threshold sufficient variations are accumulated in the library to allow the population a fast adaptation to altered conditions. Mutation rates above the error threshold after some generations bring forth, that no survivable and accordingly replicatable individuals are present anymore, und the population collapses (Eigen, M., McCaskill, J., Schuster, P.: The molecular quasispecies. Adv. Chem. Phys. 1989, 75, 149-263).

New biomolecules can be produced by a linkage of the new property to the survival or a sufficiently large growth advantage of an organism. At this the variant library is transferred into a corresponding organism and the growth conditions are chosen in a way, that only the organisms survive or comparatively grow faster, which produce a variant of the biomolecule with the wanted new property (Zaccolo, M, Gherardi, E.: The effect of high-frequency random mutagenesis on in vitro protein evolution: a study on TEM-1 beta-lactamase. J. Mol. Biol. 1999. 285, 775-83. or Samuelson, J. C., Xu, S. Y.: Directed evolution of restriction endonuclease BstYI to achieve increased substrate specificity. J. Mol. Biol. 2002. 319,673-83). This application is only applicable to a narrowly limited circle of biomolecules, which provide an advantage to a chosen organism. Biomolecules, which catalyze arbitrary chemical reactions, cannot be selected in this way. Since the organism needs to remain alive during the whole selection process, toxic or otherwise for the growth disadvantageous properties cannot be selected.

Another method for the selection of new biomolecules is the linkage of the biomolecule to the coding nucleic acid sequence (Amstutz, P., Forrer, P., Zahnd, C., Pluckthun, A.: In vitro display technologies: novel developments and applications. Curr. Opin. Biotechnol. 2001. 12. 400-5. Xia, G., Chen, L., Sera, T., Fa, M., Schultz, P. G., Romesberg, F. E.: Directed evolution of novel polymerase activities: mutation of a DNA polymerase into an efficient RNA polymerase. Proc. Natl. Acad. Sci. USA. 2002. 99. 6597-602. Pschorr, J.: Genotyp und Phänotyp koppelnde Verbindung. DE0019646372C1). An application of these technologies with living organisms like phages or bacteria limits the spectrum again to non-toxic or not growth inhibiting biomolecules. Also the substrates and products of the wanted reaction may not have any damaging effect to the presenting organism. Additionally catalytic activities can only be selected if biomolecule and substrate can be presented at the same organism. As the activity of the catalytic biomolecules cannot be limited to the organism, which presents them, and they therefore also take place reactions at other individuals of the library, this method often leads to false selection of biomolecules.

In dissection methods (screening methods) every variant of a biomolecule library is analyzed separately regarding the wanted property (Joo, H., Lin, Z., Arnold, F. H.: Laboratory evolution of peroxide-mediated cytochrome P450 hydroxylation. Nature. 1999. 399. 670-3. Korbel, G. A., Lalic, G., Shair, M. D.: Reaction microarrays: a method for rapidly determining the enantiomeric excess of thousands of samples. J. Am. Chem. Soc. 2001. 123. 361-2). Even with very short measurement times (e.g. 100 msec per variant) this methods demands a high time expense (e.g. 22 days) for the analysis of large libraries (e.g. 107). The continuous measurement of variants in these dimensions needs the setup of appropriate complex apparatuses. Besides for every variant of the library a corresponding property test needs to be run, what leads to very high costs of these methods.

To screen or to change enzymatic properties in the laboratory, the so-called “enzyme engineering”, according to the state of the art within an enzyme library genotype (a nucleic acid, which can be amplified and comprises a variant of a gene) and phenotype (a functional feature, for example a catalytic property) need to be coupled together. This coupling for instance is realized through techniques like phage display or ribosome display or thereby, that each genotype is testing individually for its phenotype.

The aim of the present invention is to give a method to identify biomolecules in variant libraries of biomolecules.

According to the present invention the aim is solved by a method for the identification of biomolecules in variant libraries of biomolecules comprising the steps:

a) Production of a variant library, consisting of a number of variants (B₀) of gene sequences coding for the biomolecule,
b) Division of the variant library into a number of compartments (W₀), which is smaller than the number of variants in the variant library (B₀) preferentially by a factor of ten, more preferentially by a factor of 100,
whereas each compartment contains a partial library which contains K₀=B₀/W₀ variants,
c) Production of biomolecules in the compartments and testing of the biomolecules obtained in the single compartments for a specified property (phenotype), preferentially a biocatalytic activity, whereas from the observed phenotype no direct conclusions on the genotype can be made,
d) Selection of at least one compartment, which contains biomolecules fulfilling the wanted property, preferentially a biocatalytic activity,
e) Division of the partial library contained in the selected library into further compartments corresponding to step b) and
f) n-fold repetition of steps c) to e) until in every compartment maximally only one variant (Kₙ<=1) of the gene sequence coding for the biomolecule is contained.

This method is especially suitable for the generation of biomolecules with new catalytic activities, which either do not exist in nature or at least cannot be catalyzed by the starting biomolecule. Furthermore with this method existing catalytic activities can be adapted to exterior conditions like for example temperature or solvent, under which no or only little activity was present.

As in the present invention the production of the biomolecules can lead to a die off of the organisms or can be carried out by cell-free systems, the method can be applied to all kind of biomolecules and is not limited to non-toxic or not growth inhibiting activities. As up to a million or more variants are analyzed with one test and simultaneously for the corresponding property, the time needed for the screening of the library and the costs needed for the property tests are reduced by a corresponding factor. Variants, which possess the wanted properties, can by isolated from the original variant mixture in a secure and reproducible way.

In the step a) of the method a variant library of gene sequences coding for the biomolecule is produced by standard molecular biology processes.

According to the present invention among a variant library is conceived: A mixture of proteins or nucleic acids, which differ from each other at least in one position of their sequence.

Preferentially the variant library consists of a number of variants in the dimension of B₀=10³ to B₀=10¹⁵. For example within a partial area of the biomolecule randomly chosen sequence modules can be introduced, so that in case of a nucleic acid with 25 altered positions a library size of 4²⁵=1.1×10¹⁵ or in case of a protein with 7 altered positions a library size of 20⁷=1.3×10⁹ originates.

More preferentially the dimension lies in the range between B₀=10⁵ to B₀=10⁹.

More preferentially the variant library consists of DNA-plasmids or linear nucleic acid molecules, which contain the gene sequence coding for the biomolecule.

According to the present invention biomolecules are proteins, nucleic acids or other biopolymers consisting of organic building blocks. Preferentially these are biomolecules, enzymes or ribozymes or other biomolecules, which as biocatalysts accelerate the conversion of chemical or biochemical substances.

Standard molecular biology methods, with which such variant library can be produced, are for example defective amplification techniques for nucleic acids. For this purpose replicating enzymes, e.g. polymerases, which conduct the novel synthesis of a biomolecule with the help of a template, are used. The introduction of mistakes and the thereby generation of different variants is achieved by the naturally existing error rate of these replicating enzymes or can be increased by changing the reaction conditions (e.g. imbalance of the synthesis building blocks, addition of building block analogues, alteration of the buffer conditions). Besides the introduction of mistakes a variant library can be obtained by using the natural occurring diversity to originate a specific biomolecule or a class of biomolecules.

In comparison to conventional screening methods the process according to the present invention allows the screening of very large libraries. The division process according to the present invention allows the simultaneous testing of an arbitrary number of variants.

The size of the library is only limited by the sensitivity of the assay, with which the biomolecules contained in the single compartments are tested for a specified property, preferentially a biocatalytic activity, in step c) of the process.

Preferentially the libraries are produced by error-prone PCR or by the introduction of synthetically randomized sequence regions (Cadwell, R. C., Joyce, G. F.: Randomization of genes by PCR mutagenesis. PCR Methods Appl. 1992. 2. 28-33; Wells, J. A., Vasser, M., Powers, D. B.: Cassette mutagenesis: an efficient method for generation of multiple mutations at defined sites. Gene. 1985. 34. 315-23).

In the process the mutation rate preferentially is chosen far beyond the error threshold. Thereby within the starting library preferentially more than 90%, more preferentially more than 99% and even more preferentially more than 99.9% of the generated variants are not survivable.

The error threshold is defined as the maximal mutation rate, which in evolutionary methods (cyclic application of mutation and selection) just not leads to a melting of the genetic information and thereby retains the survivability of a population. A melting of the genetic information is defined as a process, in which by a repeated appliance of a too high mutation rate in the replication of a nucleic acid so many mutations accumulate, that the nucleic acid does not contain any physiologically meaningful information anymore.

The survivability of a gene and accordingly a gene product is thereby defined in the way, that the gene and accordingly its gene product still is able to perform its physiological activity like for example the binding of a partner or the catalytic cleavage of a substrate.

An important advantage of the present invention in comparison to conventional methods, which contain mutagenesis and selection steps, consists therein, that in the process according to the present invention one starts from a large library, which a priori contains the wanted variant. That means that after the screening one does not obtain a suboptimal variant, which needs to be further improved through additional cycles of mutation and recombination.

The method according to the present invention is characterized thereby that in the beginning one-time in step a) a variant library is generated, which subsequently is screened for variants with the wanted property. From step b) on no additional mutation or recombination steps take place. That means that in between or during the individual singling steps (steps b. to f.) the isolated partial libraries do not undergo a further mutagenesis or recombination. That means that the variants which are isolated at the end of the process with the wanted properties are already present in the initially (in step a.) applied library.

Preferentially the process according to the present invention is conducted in a way that in step d) in all passages only one compartment is chosen namely that one in which the wanted property (phenotype) is strongest distinct, preferentially the compartment with the strongest catalytic activity. Thereby with the process according to the present invention the best variant can be isolated, in which the wanted property (phenotype) is strongest distinct, without the obligatory necessity of selecting suboptimal variants or groups of variant.

At the production of the variant library one preferentially starts from an already known nucleic acid or protein sequence, consecutively called starting sequence. Based on this starting sequence the variant library is produced by the above mentioned methods (e.g. error-prone PCR or by the introduction of synthetically randomized sequence regions).

The method according to the present invention is characterized thereby that the starting sequence does not need to be contained in the variant library.

The starting sequence often codes for a phenotype which is to a certain degree similar to the wanted property. So one would, for example when one wants to obtain an RNase as the wanted phenotype, which cleaves after an adenosine, chose for instance an RNase as the starting sequence, which cleaves after a guanosine (and not a protease or so).

However the more similar the starting sequence is to the phenotype of the wanted property the larger however is usually the background activity within the test in step c) of the process. Advantageously this background is avoided, when the starting sequence is not present in the variant library anymore.

Preferentially the variant library is produced in a way that the starting variant is not contained in the variant library anymore. This for example can be achieved thereby that a stop codon is introduced into the starting sequence, which is removed again by the introduction of mutated regions into the starting sequence. Thereby it can be assured that eventually protracted starting sequences are because of the stop codon physiologically not active and that on the other side physiologically active variants need to contain mutated regions.

In opposite to the in the state of the art applied high-throughput processes the method according to the present invention allows the screening of about multiples larger libraries in a fraction of the time. In comparison to in vivo selection methods the method according to the present invention is also not limited to specified enzyme classes and specified enzyme properties respectively.

In step b) the variant library is divided up into a number of compartments W₀, which is smaller than the number of variants contained in the variant library at least by a factor of 10, preferentially by a factor of 100.

At this before the division the variant library can be transformed into an organism or the division can be conducted on the level of the coding sequences. The division is done in a way that each variant of the library occurs at least once, preferentially exactly once.

The then in step c) conducted production (expression) of the biomolecules is done preferentially by the organism or by in vitro expression systems (e.g. cell extracts).

As expression organisms which are used regularly in molecular biology for the expression of biomolecules, like proteins, can be used, the expression organism is chosen depending on the biomolecule which needs to be expressed. Preferred expression organisms are bacterial cells (e.g. E. coli, B. subtilis) or eukaryotic cells (e.g. S. cerevisiae, insect cells, tumor cells). By the transformation of the variant library into the expression organism single clones originate. Thereby every clone contains one defined genotype respectively that is one variant of the gene sequence coding for the biomolecule. According to the present invention one clone can also be defined as a sole coding sequence that is a defined genotype without expression organism.

The transformation into an organism is done with known molecular biology methods for the transformation of gene sequences into expression organisms and depends on the expression organism used. A preferred method is electroporation.

Preferentially the division into compartments is done immediately after the transformation of the variant library into the expression organism.

The number of the compartments W₀ amounts to preferentially between 10¹ and 10⁴ compartments and more preferentially to between 96 und 1536 compartments.

The library size B₀ divided by the number of compartments W₀ gives the clone number per compartment K₀=B₀/W₀.

Every compartment contains a partial library with the number of K₀ variants of the gene sequence coding for the biomolecule.

The division particularly preferentially is done into compartments of a microtiter plate and a deep well plate respectively.

Preferentially in step c) an amplification of the partial libraries in the compartments is carried out by a growth of the organisms or by an amplification of the coding sequences by template-depending enzymes up to a number of individuals V₀ per compartment and the production of the catalytic biomolecules is carried out by the expression organisms or by cell-free expression systems like for example E. coli lysates, reticulocyte lysates, C. lucknowese lysates or insect cell lysates.

Preferentially a conservation of a part of the partial library on the level of organisms or on the level of the pure coding sequences at the point in time×under retention of the compartment allocation is carried out.

The conservation is carried out preferentially by the production of a 1:1 mixture of the organism culture and glycerol and storing of that mixture under growth inhibition at −80° C. A conservation on the level of the coding sequences is carried out by taking off a part of the amplified sequences and storage, preferentially at −20° C.

A determination of the number of individuals V₀(x) of the conserved partial library on the level of organisms is preferentially carried out by measuring the optical density OD of a liquid organism culture and correlation with the number of individuals or by transferring an aliquot of this culture to a solid medium and counting the thereof resulting colonies. The determination of the number of individuals V₀(x) of the conserved partial library on the level of the coding sequences is carried out preferentially by determining the concentration with spectroscopic methods.

The number of individuals V₀(x) divided by the number of clones per compartment K₀ gives the amplification factor F₀(x) per clone, F₀(x)=V₀(x)/K₀.

In step c) of the process the biomolecules contained in the single compartments are tested for a specified property (phenotype), preferentially for a biocatalytic activity.

In step c) an amplification of the partial library in the compartments is preferentially carried out up to a number of individuals V₀(x) at the point in time×per compartment, whereas the number of individuals divided by the number of clones per compartment K₀ gives the amplification factor F₀(x) per clone.

Before, during or after the growth of the organisms or the amplification of genotypes the production of the biomolecules is carried out thereby in the single compartments.

Preferentially the test is carried out for a biocatalytic activity by incubating the catalytically active biomolecules contained in the compartments or isolated from them with corresponding substrates and allocating activity values to the corresponding compartments. Compartments, in which the activity value exceeds a defined barrier, are assessed as positive.

As each compartment contains more than one clone of the variant library, no conclusion can be made from the observed phenotype to the genotype, because the observed phenotype results from the sum of clones contained in the compartment.

Although therefore in the method according to the present invention genotype and phenotype are decoupled, the clone responsible for the wanted property, which for instance comprises the wanted enzymatic activity, can be retrieved and isolated from the mixture of clones with the method according to the present invention. That it is possible to retrieve the clone responsible for the wanted property from the mixture of clones with a screening method, in which genotype and phenotype are decoupled, is surprising to persons skilled in the art, as all known screening methods base on the coupling of genotype and phenotype.

To retrieve the clone with the wanted property is achieved with the steps d) and e) of the method according to the present invention.

In step d) of the process at least one compartment is chosen, which contains biomolecules, which fulfill the wanted properties.

Preferentially therefore the partial library or the corresponding conserved partial library is diluted by the means of factor F₀(x), so that in a given volume each clone contained in the compartment statistically occurs up to a number of X₀<W. This volume in turn is divided up into a number W₁ of new compartments without a prior amplification. The new number of clones per compartment is K₁=X₀*K₀/W₁.

Now the steps c) to e) of the process are repeated as often as the number of clones per compartment Kₙ≦1. As soon as Kₙ≦1, the wanted phenotype can be allocated to a discrete genotype.

In order to avoid the loss of single clones and thus of variants of the library of biomolecules, the step e) preferentially is conducted in a way that in the first passages of steps e) 1<Xₙ₋₁<W₁ applies, preferentially Xₙ₋₁=3 to 5.

Step e) preferentially is repeated as often as the clone causing the wanted property is to be found in the new compartmented partial library. At this in the last passage of step e) Xₙ preferentially is <1. Therefore the partial library preferentially is diluted in the last passage of step e) in a way that maximally one clone can be found per compartment and that in many compartments no clone is contained. Therewith an average number of Xₙ<1 results.

In step f) the steps c) to e) are repeated n-fold until in each compartment maximally only one variant (Kₙ<=1) of the gene sequence coding for the biomolecule is contained.

Die number of necessary repetitions n is depending on the number of variants (B₀) of the in step a) constituted variant library, the number of compartments (Wₙ) in which the library is divided up in step b) and e) und the number Xₙ, with which a once retrieved clone will again be present in the next cycle. The number of conducted repetitions n thereby amounts to with a preferentially constant Xₙ=1 and constant Wₙ:

n=log₁₀(B₀)−log₁₀(Wₙ) oder n=(log₁₀(B₀)−log₁₀(Wₙ))+1,

whereas n eventually is rounded up to the next larger whole number.

If in step a) for example a library with B₀=10⁶ variants is constituted und if the partial libraries in step b) and e) are divided up with Xₙ=1 in Wₙ=96 or Wₙ=100 compartments respectively, than n=4 to 5 passages of the steps c) to e) are necessary in order to retrieve the clone with the wanted property.

With the consecutive execution examples the invention is illustrated in detail:

Execution example 1 describes exemplarily the selection of active RNase T1 from a variant library of inactive variants of RNase T1.

Execution example 2 describes exemplarily the selection of an adenosine cleaving RNase T1 from a library of RNase T1 variants.

### EXECUTION EXAMPLE 1

### 1. Cloning the Genes of RNase T1 Wildtype and His92Ala

With the two primers A2Vo_BspHI (SEQ_ID No. 1) and A2Hi_PstI (SEQ_ID No. 2) (both from IBA Goettingen, Germany) the genes coding for RNase T1 wildtype (SEQ_ID No. 3) and for RNase T1 variant His92Ala (SEQ_JD No. 4) including the signal peptide for a periplasmatic expression were amplified from the corresponding source vectors pA2T1 (SEQ_ID No. 5) und pA2T1_H92A (SEQ_ID No. 5, in which SEQ_ID No. 3 is replaced by SEQ_ID No. 4) by a PCR under the following conditions:

### 1.1 PCR:

1.1 PCR:

| | | | | |
|---|---|---|---|---|
| PCR-reaction: | 10 | μl | 10× VENT-buffer (NEB, Beverly, USA) | |
| | 2 | μl | dNTPs (each 10 mmol/liter) | |
| | 100 | pmol | Primer A2Vo_BspHI | (SEQ_ID No. 1) |
| | 100 | pmol | Primer A2Hi_PstI | (SEQ_ID No. 2) |
| | 1 | μl | original vector (20 ng) | (SEQ_ID No. 5) |
| | 2 | U | VENT-Polymerase (NEB) | |
| | ad 100 | μl | H₂O dest. | |

| PCR temperature profile: | 2 min/94° C. | | | |
|---|---|---|---|---|
| | 1. | 45 sec/94° C. (denaturation) | | |
| | 2. | 45 sec/57° C. (annealing) | {close oversize brace} | 25× |
| | 3. | 30 sec/72° C. (elongation) | | |

2 min/72° C.

The resulting PCR-products were purified with the QIAquick PCR-purification-kit (Qiagen, Hilden, Germany) following the manufacturers instructions.

### 1.2 Restriction Digest:

In order to clone the genes into the expression vector pETBlue-2 (SEQ_ID No. 6) the PCR-products and the vector were incubated with restriction endonucleases BspHI and PstI and NcoI and PstI (all from MBI Fermentas, Vilnius, Lithuania) respectively as follows:

### Restriction Digest Reactions:

| PCR-Products: | | | Vector: | | |
|---|---|---|---|---|---|
| 2 | μg | PCR-product | 4 | μg | pETBlue-2 |
| 2 | μl | 10x buffer O⁺ (MBI) | 2 | μl | 10x buffer Y⁺ (MBI) |
| 10 | U | BspHI | 10 | U | NcoI |
| 10 | U | PstI | 10 | U | PstI |
| ad 20 | μl | H₂O dest. | ad 20 | μl | H₂O dest. |

The restriction digest reactions were incubated for 2 h at 37° C. To the “vector-reaction” subsequently for the dephosphorylation 1 U SAP (MBI Fermentas, Vilnius, Lithuania) is added and incubated for additional 30 min at 37° C. Afterwards the enzymes get inactivated for 20 min at 80° C. Hereupon the products are purified with the QIAquick PCR-purification-kit (Qiagen, Hilden, Germany).

1.3 Ligation, Transformation into E. Coli and Plasmid-Preparation

The vector-DNA and the PCR-product are ligated by the incubation with T4-DNA-ligase as follows:

| | | | |
|---|---|---|---|
| Ligase-reaction: | 200 | fmol | Vector-DNA |
| | 600 | fmol | PCR-Product |
| | 3 | μl | 10x Ligase-buffer (MBI) |
| | 1 | μl | T4-DNA-ligase |
| | ad 30 | μl | H₂O dest. |

The reactions are incubated for 8 h at 16° C. and the enzyme is subsequently inactivated by a 10 minute incubation at 65° C. 1 μl of this reaction was directly used for the transformation of commercially available competent ElectroTen-cells (Stratagene, La Jolla, USA) with electroporation. The electroporated cells were plated on agar plates with ampicillin and cultivated over night at 37° C. Starting from a resulting single colony the ready plasmid was re-isolated with the plasmid-purification kit QIAprep Minipreparation-kit (Qiagen, Hilden, Germany) following the manufacturers instructions.

### 1.4 Production of a Plasmid Mixture as RNase T1-Test Library:

As result from the preceding steps the two plasmids pETBlue-RNaseT1-wildtype and pETBlue-RNaseT1-His92Ala are obtained.

In order to produce the test library the plasmid are mixed as follows:

1 pg pETBlue-RNaseT1-wildtype is mixed with 1 μg pETBlue-RNaseT1-His92Ala. Thereby one obtains a relation of 1:1,000,000 RNase T1 wildtype (active) to the variant His92Ala (inactive).

### 1.5 Production of the Expression Strain:

For the expression of the RNase T1-test library an E. coli strain is needed, in which the RNase I is knocked out. Corresponding strains like for example AT9 (rna⁻19λ⁻ gdhA2 relA1 spoT1 metB1) are available via the E. Coli Genetic Stock Center New Haven, USA. The expression vector pETBlue-2 used in the example additionally needs the T7-RNA-polymerase for the expression, which is not present in E. coli. With the commercially available λDE3-Lysogenisation-kit (Novagen, Madison, USA) the T7-RNA-polymerase coding gene is introduced into the strain AT9. Through this an E. coli-strain is obtained, which is characterized by the absence of RNase I and the presence of the T7-RNA-polymerase (DE3). Electrocompetent cells were prepared from this strain with standard molecular biology methods and stored at −80° C.

1.6 Transformation of the Expression Strain with the Test Library:

Into the strain produced as precedent described one ng of the plasmid mixture as a test library was transformed via electroporation and the resulting cells were taken up into 10 ml liquid medium (LB-medium: 10 g Tryptone, 5 g yeast extract (all from Becton Dickinson, Heidelberg, Germany), 10 g NaCl (from Sigma, Deisenhofen, Germany)) containing ampicillin after 1 hour incubation at 37° C.

The in this way obtained preparatory culture is immediately divided on a 96-well microtiter plate (MTP) (100 μl per well) and incubated at 30° C. and 800 rpm over night.

By the transformations with electroporation approximately 3 million transformed clones are obtained.

### 1.7 Growth of the Main Culture and Expression of RNase T1

A 96-well deep well plate (DWP) is filled with 1.5 ml liquid medium with ampicillin per well respectively. The medium is inoculated with 50 μl from the preparatory culture respectively and the DWP is cultured at 37° C. and 800 rpm. When an optical density OD₆₀₀ of the cultures of OD₆₀₀=1.0 is reached the cultures are induced with 1 mmol/liter IPTG. Afterwards the plate is incubated for additional 4 h at 37° C. and 800 rpm.

### 1.8 Preparation of Protein Samples

By the signal peptide ompA the expressed RNase T1-molecules are directed into the periplasmatic space of the expression bacterium. Through an osmotic shock the protein can be prepared very easily. The purification procedure comprises the following steps:
Collection of the cells by centrifugation at 4000 rpm, 4° C. for 5 min,
Decantation of the medium supernatant,
Resuspension of the bacterial pellet in 25 μl buffer A (50 mmol/liter Tris/HCl, pH 7.5, 10 mmol/liter EDTA, 15% Saccharose w/v) respectively,
Incubation on ice for 30 min,
Addition of 125 μl buffer B (50 mmol/liter Tris/HCl, pH 7.5, 10 mmol/liter EDTA) respectively,
Centrifugation at 4000 rpm, 4° C., for 20 min,
Removal of the supernatant and transfer into a MTP (periplasm),
Storage of the bacterial pellet.

### 1.9 Production of the Substrate for RNase T1

As a substrate (Sub_G) a double stranded DNA-molecule with a central single stranded area was used, which contained a guanosine-RNA-Building block as point of attack for the enzyme. The ends of this substrate are labeled with differing dyes for the red (Cy5 at the 5′-end) and the green (RhG at the 3′-end) spectral range. In order to avoid a bleaching of the labeled substrate the corresponding solutions and incubation reactions are protected from light. The buffers and reactions were produced with DEPC-treated water. The substrate is composed of the following three oligonucleotides (IBA Goettingen, Germany):

1. Sub_G:

(SEQ_ID No. 10)

5′-Cy5-CCATACCAGCCAGCCACAArGCAAGCCACCGAAGCACAGATA-
RhG-3′
2. T1_Sub_Li:

(SEQ_ID No. 7)

5′-GTGGCTGGCTGGTATGGA-3′
3. T1_Sub_Re:

(SEQ_ID No. 8)

5′-TATCTGTGCTTCGGTGGC-3′

By the consecutively described hybridisation the three components are annealed to a double stranded substrate:

| Hybridisation reaction: | | | Hybridisation program: |
|---|---|---|---|
| 1000 | pmol | Sub_G | 1. 10 sec 94° C.; |
| 1200 | pmol | T1_Sub_Li | 2. Cooling to 25° C. with 0.1° C./sec |
| 1200 | pmol | T1_Sub_Re | 3. 4° C. |
| 20 | μl | MES (1 mol/liter, | |
| | | pH 6.0) | |
| ad 1000 | μl | DEPC-H₂O | |

1.10 Incubation of the Protein Samples with the Substrate

In a MTP 10 μl of the double stranded substrate are provided per well respectively. Thereto 10 μl of the protein samples isolated from the periplasm are added respectively, the MTP is sealed air-proof and incubated for 24 h at 37° C. in the dark. Afterwards 5 μl of the reactions are transferred into a MTP with glass bottom respectively and mixed with 250 μl buffer C respectively (100 mmol/liter MES, pH 6.0, 100 mmol/liter NaCl, 2 mmol/liter EDTA).

### 1.11 Activity Determination

In order to determine the enzyme activity the plate with the glass bottom, into which the incubation reactions were transferred as described in 1.10, was measured on the fluorescence correlation spectroscope ConfoCor 2 (Evotec Biosystems, Hamburg, Germany and Carl Zeiss Microscopy, Jena, Germany). The evaluation of the date was conducted using the ConfoCor 2-software (version 2.5).

For the measurements an Argon-laser (1=488 nm) is used for the excitation of RhG in combination with a helium/neon-laser (1=633 nm) for Cy5. The FCS measurement volume in the cavities was adjusted 200 μm above the glass surface. The measurements were conducted for 20 sec per well.

By a cross correlation analysis of the obtained data one can conclude on an eventual cleavage of the substrate. A cleavage of the substrate by RNase T1 leads to a decoupling of both fluorescent dyes and therefore to a loss of the cross correlation signal. Uncut substrate molecules in contrast carry both dyes and deliver a strong signal.

By the division of the 3 million clones obtained by transformation und through the mixture relation between active RNase T1 wildtype and inactive RNase T1 His92Ala of 1:1,000,000 theoretically three wells with activity should be detectable with measurements. Statistical deviations between 1 to 5 wells with activity are however possible.

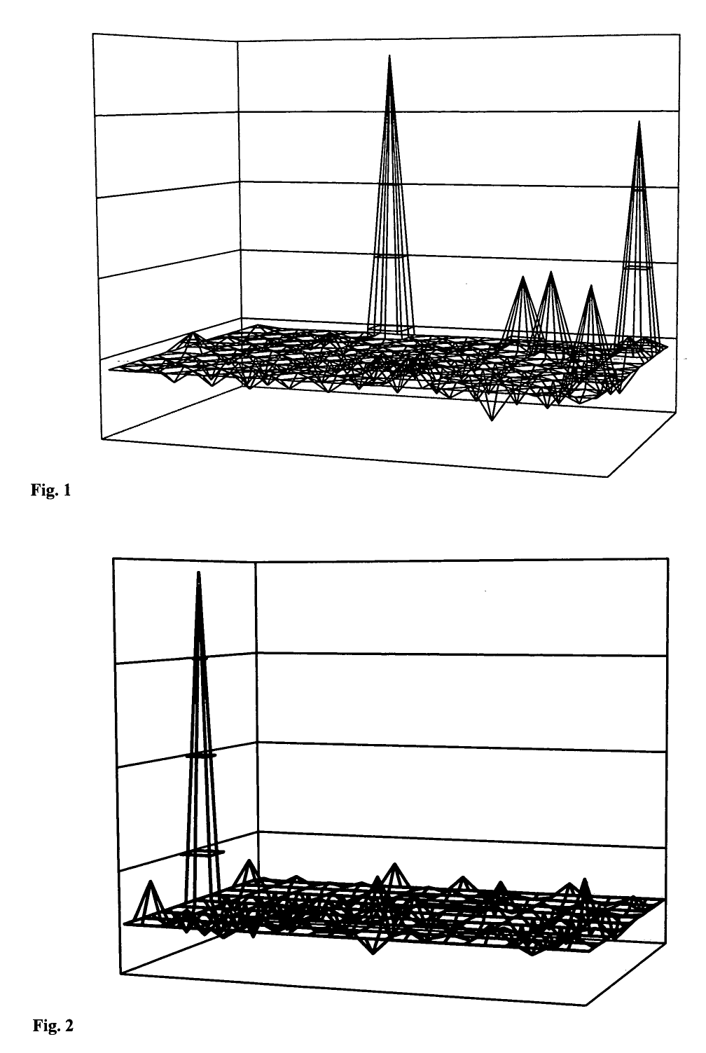

FIG. 1 shows the thus obtained data for an RNase T1-test library produced as described in point 1 to 1.11 consisting of 3 million clones on one plate with a mixture relation of RNase T1-wildtype to RNase T1-His92Ala of 1:1,000,000. The RNase T1-activity was detected as described above via cross correlation analysis. For a better overview a reciprocal view was chosen, that means that high peaks mean a low signal and low peaks a high signal. FIG. 1 shows 2 clear peaks, which are caused by a loss of the cross correlation signal. These two peaks indicate that in the experiment an RNase T1-activity in two of 96 wells securely was present.

### 2. Re-Isolation of the Partial Library

In the plate obtained in section 1 a plasmid preparation is conducted with the stored bacterial pellets from the protein preparation using the QIAprep Minipreparation-kit (Qiagen, Hilden, Germany) with one of the wells, which showed an RNase T1-activity in the activity measurement (section 1.11),

By the original division of 3 million clones on the plate a number of 3,000,000/96=31,250 different clones per well resulted. Therefore a mixture relation from RNase T1 wildtype to RNase T1 His92Ala of 1:32,250 consists in the isolated partial library.

### 2.1 Additional Separatings

Through a transformation of different aliquots of the thus obtained partial library in analogy to section 1.6 the amount of plasmid DNA was determined, which is necessary to now obtain about 100,000 transformed clones via electroporation.

Afterwards the determined amount of the partial library is transformed into the expression strain and the same process as for the test library is conducted. As about 100,000 clones were divided up and the new mixture relation was 1:32,250, again theoretically three wells with detectable activity were expectable.

The plasmids were again re-isolated from the bacterial pellets from one of the wells with activity. The mixture relation in this again enriched partial library was now 100,000/96=1,050.

An additional repetition of the depicted scheme with a division of now about 3,000 clones gave a once again enriched partial library with a mixture relation of 3,000/96=31.

As from this last partial library 96 clones were subdivided on a MTP, three wells resulted with activity. As these activities now resulted from an individual clone respectively, the activity of RNase T1 wildtype could be directly allocated to this clone.

### EXECUTION EXAMPLE 2

Wildtype RNase T1 cleaves RNA in a highly specific way after guanosine residues. The aim of this execution example is to obtain RNase T1 variants which can cleave RNA at adenosine residues. Therefore an RNase Ta library was produced and screened for corresponding variants.

### 1. Design of the Library

The region of the guanosine binding loop 1 which needed to be mutagenized comprises the amino acids 41 to 57 of RNase T1 wildtype (SEQ_ID No. 3). The loop 1-DNA-sequence is mutated by a corresponding synthesized mutagenesis-oligodesoxynucleotide Loop 1_{—}32 in a way that 3 to 4 of the 17 amino acids respectively are randomly replaced by others. Therefore the following sequence is synthesized:

5′-GTAGGATCCAATTCTTACCCACAC aay tax aax aax tax
gay ggz ttz gaz ttx tcz gty agx tcz ccx tax tax
GAATGGCCTATCCTCTCGAGCGG-3′

in which “n” (A, G, C or T—“any”) and “b” (G, C or T—not A) from SEQ_ID No. 9 are precisely defined as follows:
a=86% A 6% C 4% G
   x=c′=88% C 6% G 6% T
   c=86% C 6% A 4% G
   y=g′=82% G 11% C 7% T
   g=79% G 8% A 8% C
   z=t′=82% T 11% C 7% G
   t=79% T 8% A 8% C

### With A=Adenine, C=Cytosine, G=Guanine, T=Thymine.

The oligonucleotide Loop1_{—}32 (IBA, Goettingen, Germany) is afterwards directly used as a primer (in section 3.1) in a PCR.

### 2. Production of the Vector for the Screening

The gene of RNase T1 wildtype (SEQ_ID No. 3) including the signal peptide for a periplasmatic expression is cloned into the vector pETBlue-2 (Seq_ID No. 6) as described in the execution example 1 (section 1.1.-1.3.) and the vector pETBlue-RNase T1-wildtype is obtained.

Afterwards the vector pETBlue-RNase T1-wildtype is digested with PvuII und SspI (both from MBI Fermentas, Vilnius, Lithuania):

### Reaction:

| | | |
|---|---|---|
| 4 | μg | pETBlue-2 |
| 2 | μl | 10x buffer G (MBI) |
| 10 | U | SspI |
| 10 | U | PvuII |
| ad 20 | μ1 | H₂O dest. |

The restriction digest reaction is incubated for 2 h at 37° C. Afterwards the enzymes are inactivated for 20 min at 80° C. The products are separated on a 0.8% agarose gel and the product band at 2498 bp is cut out from the gel. The DNA is consecutively re-isolated via the QIAquick gel-extraction-kit (Qiagen, Hilden, Germany). 200 fmol of the isolated fragment are recircularized in a ligation:

| | | |
|---|---|---|
| Reaction: | 200 fmol | fragment |
| | 2 μl | 10x Ligase-buffer (MBI) |
| | 2 μl | 50% PEG (MBI) |
| | 1 μl | T4-DNA-Ligase |
| | ad 20 μl | H₂O dest. |

The reactions are incubated for 8 h at 16° C. and the enzyme is subsequently inactivated by a 10 minute incubation at 65° C. 1 μl of this reaction was directly used for the transformation of commercially available competent ElectroTen-cells (Stratagene, La Jolla, USA) with electroporation. The electroporated cells were plated on agar plates with ampicillin and cultivated over night at 37° C. Starting from a resulting single colony the ready plasmid was re-isolated with the plasmid-purification kit QIAprep Minipreparation-kit (Qiagen, Hilden, Germany) following the manufacturers instructions. The thereby obtained plasmid is named pETMini_RNaseT1_wildtype.

### 3. Cloning of the Library RNaseT1-Loop1

With the both primers Loop1_{—}32 (SEQ_ID No. 9) and A2Hi_PstI (SEQ_ID No. 2) (both from IBA Goettingen, Germany) a part of the RNase T1 Wildtyp (SEQ_ID No. 3) is amplified from the original vector pA2T1 (SEQ_ID No. 5) through a PCR under the following conditions:

### 3.1 PCR:

3.1 PCR:

| | | | | |
|---|---|---|---|---|
| PCR-reaction: | 10 | μl | 10× Taq-buffer (MBI Fermentas, Vilnius, Lithuania) | |
| | 2 | μl | dNTPs (each 10 mmol/liter) | |
| | 100 | pmol | primer Loop1_32 | (refer to section 1) |
| | 100 | pmol | primer A2Hi_PstI | (SEQ_ID No. 2) |
| | 1 | μl | original vector (20 ng) | (SEQ_ID No. 5) |
| | 2 | U | Taq-polymerase (MBI) | |
| | ad 100 | μl | H₂O dest. | |

| Temperature profile of the PCR: | 2 min/94° C. | | | |
|---|---|---|---|---|
| | 1. | 45 sec/94° C. (denaturation) | | |
| | 2. | 45 sec/57° C. (annealing) | {close oversize brace} | 30× |
| | 3. | 30 sec/72° C. (elongation) | | |

2 min/72° C.

The resulting PCR-products were purified with the QIAquick PCR-purification-kit (Qiagen, Hilden, Germany) following the manufacturers instructions.

### 3.2 Restriction Digest:

To clone the library into the expression vector pETMini_RNaseT1_wildtype the PCR product and the vector are incubated using the restriction endonucleases BamHI and PstI (both from MBI Fermentas, Vilnius, Lithuania) as follows:

### Restriction Digest Reactions:

| PCR-Product: | | | Vector: | | |
|---|---|---|---|---|---|
| 2 | μg | PCR-product | 4 | μg | pETMini_RNaseT1_wildtype |
| 2 | μl | 10x buffer G⁺ | 2 | μl | 10x buffer G⁺ (MBI) |
| | | (MBI) | | | |
| 10 | U | BamHI | 10 | U | BamHI |
| 10 | U | PstI | 10 | U | PstI |
| ad 20 | μl | H₂O dest. | ad 20 | μl | H₂O dest. |

The restriction digest reactions are incubated for 2 h at 37° C. To the “vector-reaction” subsequently for the dephosphorylation 1 U SAP (MBI Fermentas, Vilnius, Lithuania) is added and incubated for additional 30 min at 37° C. Afterwards the enzymes get inactivated for 20 min at 80° C. The products are separated on a 0.8% agarose gel and for the vector reaction the product band at 2608 bp and for the PCR-reaction the product band at 259 bp is cut out from the gel. The DNA is consecutively re-isolated from the gel pieces via the QIAquick gel-extraction-kit (Qiagen, Hilden, Germany).

3.3 Ligation, Transformation into E. coli and Plasmid-Re-Isolation

The vector DNA and the PCR product are connected with T4-DNA-Ligase as follows:

| | | | |
|---|---|---|---|
| Ligase-reaction: | 200 | fmol | Vector-DNA |
| | 600 | fmol | PCR-product |
| | 3 | μl | 10x Ligase-buffer (MBI) |
| | 1 | μl | T4-DNA-Ligase |
| | ad 30 | μl | H₂O dest. |

The reactions are incubated for 8 h at 16° C. and subsequently the enzyme was inactivated by a 10 minute incubation at 65° C. The enzymes are removed from the solution by shaking out with phenol/chloroform twice and the obtained aqueous solution is precipitated by adding the 2.5-fold volume of ethanol and incubation for 1 h at −20° C. The reaction subsequently is centrifuged for 15 minutes with 15,000 rpm at 4° C. and the pellet is washed with 70% ethanol. After an additional 15 minute centrifugation at 13,000 rpm at 4° C. the ethanol is taken off and the DNA-pellet is dried. Afterwards the DNA is resolved in 3 μl H₂O dest. and directly used for the transformation of commercially available competent ElectroTen-cells (Stratagene, La Jolla, USA) via electroporation. From the electroporated cells 10 μl are plated on agar plates with ampicillin and incubated at 37° C. The rest of the electroporated cells is directly diluted into 100 ml liquid medium (LB-medium: 10 g Tryptone, 5 g yeast extract (both from Becton Dickinson, Heidelberg, Germany), 10 g NaCl (Sigma, Deisenhofen, Germany)) containing ampicillin and also incubated over night. The colonies on the agar plate are counted and from the value the total size of the whole library is determined. Starting from 5 ml of the liquid culture, in which the clone mixture has grown, the ready plasmid library is isolated with the plasmid purification kit QIAprep Mini-preparation-7kit (Qiagen, Hilden, Germany) following the manufacturer's instructions. As the result one obtains a library of up to 10⁷ different RNaseT1_Loop1-variants: pETMini_RNaseT1_L1.

### 3.4 Production of the Expression Strain:

For the expression of the RNase T1-test library an E. coli strain is needed, in which the RNase I is knocked out. Corresponding strains like for example AT9 (rna⁻19λ⁻ gdhA2 relA1 spoT1 metB1) are available via the E. coli Genetic Stock Center New Haven, USA. The expression vector pETBlue-2 used in the example additionally needs the T7-RNA-polymerase for the expression, which is not present in E. coli. With the commercially available λDE3-Lysogenisation-kit (Novagen, Madison, USA) the T7-RNA-polymerase coding gene is introduced into the strain AT9. Through this an E. coli-strain is obtained, which is characterized by the absence of RNase I and the presence of the T7-RNA-polymerase (DE3). Electrocompetent cells were prepared from this strain with standard molecular biology methods and stored at −80° C.

3.5 Transformation of the Expression Strain with the Library:

Into the strain produced as precedent described 1 ng of the library pETMini_RNaseT1_L1 was transformed via electroporation and the resulting cells were taken up into 200 ml liquid medium (LB-medium: 10 g Tryptone, 5 g yeast extract (all from Becton Dickinson, Heidelberg, Germany), 10 g NaCl (from Sigma, Deisenhofen, Germany)) containing ampicillin after 1 hour incubation at 37° C.

10 ml of the thus obtained preparatory culture are immediately divided onto a 96 well microtiterplate (MTP) (100 μl per well) and incubated at 30° C. and 800 rpm overnight.

Thereby about 150,000 clones are obtained on the MTP.

### 3.6 Growth of the Main Culture and Expression of Rnase T1

A 96-well deep well plate (DWP) is filled with 1.5 ml liquid medium with ampicillin per well respectively. The medium is inoculated with 50 μl from the preparatory culture respectively and the DWP is cultured at 37° C. and 800 rpm. When an optical density OD₆₀₀ of the cultures of OD₆₀₀=1.0 is reached the cultures are induced with 1 mmol/liter IPTG. Afterwards the plate is incubated for additional 4 h at 37° C. and 800 rpm.

### 3.7 Preparation of Protein Samples

By the signal peptide ompA the expressed RNase T1-molecules are directed into the periplasmatic space of the expression bacterium. Through an osmotic shock the protein can be prepared very easily. The purification procedure comprises the following steps:
Collection of the cells by centrifugation at 4000 rpm, 4° C. for 5 min,
Decantation of the medium supernatant,
Resuspension of the bacterial pellet in 25 μl buffer A (50 mmol/liter Tris/HCl, pH 7.5, 10 mmol/liter EDTA, 15% Saccharose w/v) respectively,
Incubation on ice for 30 min,
Addition of 125 μl buffer B (50 mmol/liter Tris/HCl, pH 7.5, 10 mmol/liter EDTA) respectively,
Centrifugation at 4000 rpm, 4° C., for 20 min,
Removal of the supernatant and transfer into a MTP (Periplasm),
Storage of the bacterial pellet.

### 3.8 Production of The Substrate for Rnase T1

As a substrate (Sub_A) a double stranded DNA-molecule with a central single stranded area was used, which now contains an adenosine-RNA-Building block as point of attack for the enzyme. The ends of this substrate are labeled with differing dyes for the red (Cy5 at the 5′-end) and the green (RhG at the 3′-end) spectral range. In order to avoid a bleaching of the labeled substrate the corresponding solutions and incubation reactions are protected from light. The buffers and reactions were produced with DEPC-treated water. The substrate is composed of the following three oligonucleotides (IBA Goettingen, Germany):

1. Sub_A:

(SEQ_ID No. 11)

5′-Cy5-CCATACCAGCCAGCCACAArACAAGCCACCGAAGCACAGATA-
RhG-3′
2. T1_Sub_Li:

(SEQ_ID No. 7)

5′-GTGGCTGGCTGGTATGGA-3′
3. T1_Sub_Re:

(SEQ_ID No. 8)

5′-TATCTGTGCTTCGGTGGC-3′

By the consecutively described hybridisation the three components are annealed to a double stranded substrate:

| Hybridisation reaction: | | | Hybridisation program: |
|---|---|---|---|
| 1000 | pmol | Sub_A | 1. 10 sec 94° C.; |
| 1200 | pmol | T1_Sub_Li | 2. Cooling to 25° C. with 0.1° C./sec |
| 1200 | pmol | T1_Sub_Re | 3. 4° C. |
| 20 | μl | MES (1 mol/liter, | |
| | | pH 6.0) | |
| ad 1000 | μl | DEPC-H₂O | |

3.9 Incubation of the Protein Samples with the Substrate

In a MTP 10 μl of the double stranded substrate are provided per well respectively. Thereto 10 μl of the protein samples isolated from the periplasm are added respectively, the MTP is sealed air-proof and incubated for 24 h at 37° C. in the dark. Afterwards 5 μl of the reactions are transferred into a MTP with glass bottom respectively and mixed with 250 μl buffer C respectively (100 mmol/liter MES, pH 6.0, 100 mmol/liter NaCl, 2 mmol/liter EDTA).

### 3.10 Activity Determination

In order to determine the enzyme activity the plate with the glass bottom, into which the incubation reactions were transferred as described in 1.10, was measured on the fluorescence correlation spectroscope ConfoCor 2 (Evotec Biosystems, Hamburg, Germany and Carl Zeiss Microscopy, Jena, Germany). The evaluation of the date was conducted using the ConfoCor 2-software (version 2.5).

For the measurements an Argon-laser (1=488 nm) is used for the excitation of RhG in combination with a helium/neon-laser (1=633 nm) for Cy5. The FCS measurement volume in the cavities was adjusted 200 μm above the glass surface. The measurements were conducted for 20 sec per well.

By a cross correlation analysis of the obtained data one can conclude on an eventual cleavage of the substrate. A cleavage of the substrate by RNase T1 leads to a decoupling of both fluorescent dyes and therefore to a loss of the cross correlation signal. Uncut substrate molecules in contrast carry both dyes and deliver a strong signal.

FIG. 2 shows the thus obtained measurement data for a RNaseT1_Loop1-library produced according to the execution example 2 consisting of 150,000 clones on one plate. The RNase T1-activity was detected as described above via cross correlation analysis. For a better overview a reciprocal view was chosen, i.e. that high peaks mean a low signal and low peaks a high signal. FIG. 2 shows 1 clear peak, which is caused by a loss of the cross correlation signal. This peak indicates that in the experiment an RNase T1-activity, which now is able to cut a substrate after A, was present in one of the 96 wells.

### 4. Re-Isolation of the Partial Library

In the plate obtained according to execution example 2 (section 1.-3.10.) a plasmid preparation is conducted with the stored bacterial pellet from the protein preparation of the well, in which the activity determination (3.10.) has shown an RNase T1-activity after adenosine, using the QIAprep Mini-preparation-kit (Qiagen, Hilden, Germany).

Through the original division of 150,000 clones on the plate a number of 150,000/96=1563 different clones per well resulted.

### 5.1 Further Separations—1. Step

Through a transformation of different aliquots of the thus obtained partial library analogous to the execution example 1 (section 1.6) the amount of plasmid DNA was determined, which is necessary, to now obtain 5,000 transformed clones via electroporation.

Afterwards the determined amount of the partial library was transformed into the expression strain and the same process as for the original library is conducted.

As in the original well 1563 different clones were present und about 5000 clones were divided up, it should be possible to find the adenosine-cleaving activity showing clone about 3 times.

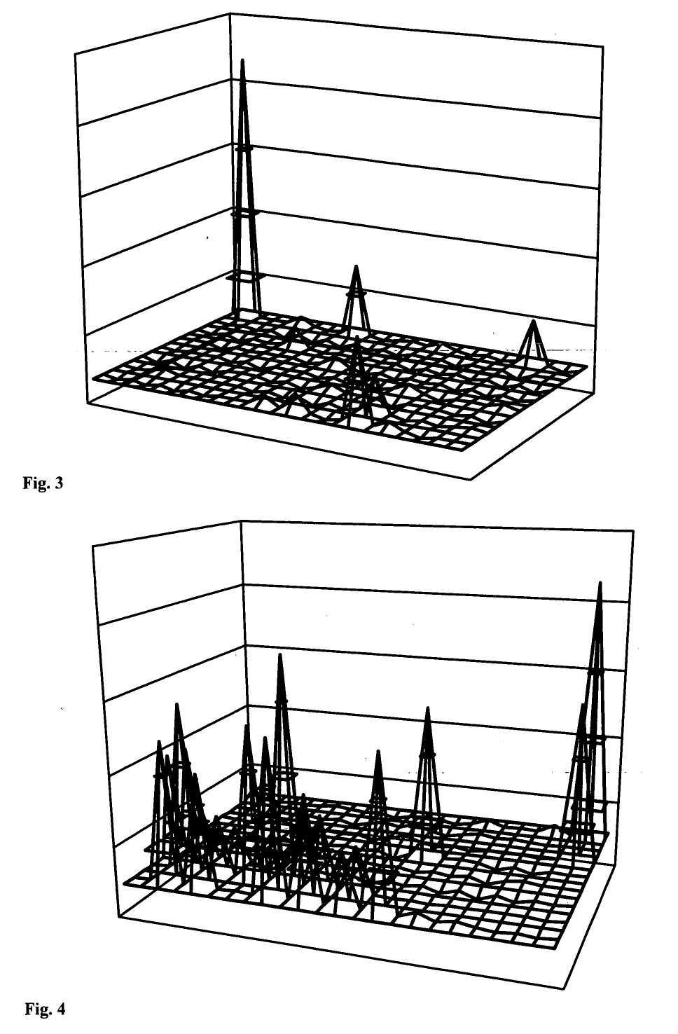

FIG. 3 shows the obtained data fort his partial library. One well was detected with a very high activity and three additional were detected with an activity which can be clearly distinct from the background, so that the clone was present 4 times in the plate. The well with the highest activity value was chosen for the additional singling step. In this well no more than 5000/96=52 different clones were present. The plasmids in turn were re-isolated from the bacterial pellet in this well.

### 5.2 Additional Separations—2. Step

An additional repetition of the depicted scheme with a division of now about 500 clones lead to an additional enriched partial library of in average 250/96=5.2 clones per well. The activity producing clone could be re-found on this plate 10 times (FIG. 4). From one of the activity showing wells again the plasmids were isolated from the bacterial pellet.

### 5.3 Additional Separations—3. Step

An aliquot of the plasmid mixture was electroporated into the expression strain and the transformants were plated on an agar plate and the plate was incubated at 37° C. overnight. From the grown single colonies 20 were selected and therewith 100 μl of preparatory culture were directly put forth on a MTP like in 3.5. After conducting the steps 3.6-3.10 the detected activity could be allocated to a single clone and the genotype of the adenosine-cleaving RNaseT1-variant could be identified.

### LIST OF ABBREVIATIONS

In the description of the invention the following abbreviations are used:
B. subtilis Bacillus subtilis
C. lucknowese Chrysosporium lucknowese
Cy5 Fluorescence dye Cy5™ (Amersham Biosciences UK Limited, Little Chalfont, Buckinghamshire, GB)
DEPC Diethyl pyrocarbonate
DWP Deep well plate
E. coli Eschericha coli
EDTA Ethylene diamine tetra acetic acid
h hour
IPTG Isopropyl-β-D-thiogalacto-pyranoside
LB Luria Broth
MES Morpholinoethane sulfonic acid
min minutes
MTP microtiter plate
OD optical density
OD₆₀₀ optical density at 600 nm
ompA outer membrane protein A from E. coli
p plasmid
PCR polymerase-chain-reaction
PT7 T7-promotor
rA Riboadenylic acid residue
rG Riboguanylic acid residue
rpm rounds per minute
RhG Rhodamine Green (Fluorescence dye)
SAP Alkaline phosphatase from shrimp
S. cerevisiae Saccharomyces cerevisiae (yeast)
Tris Tris-(hydroxymethyl)-aminomethane
T4 coming from bacteriophage T4
U Unit (for enzyme activity)
w/v weight per volume

1. Method for the identification of biomolecules in variant libraries of biomolecules comprising the steps:
a) Production of a variant library, consisting of a number of variants (B₀) of gene sequences coding for the biomolecule, and
b) Division of the variant library into a number of compartments (W₀), which is at least by a factor of ten smaller than the number of variants in the variant library (B₀),
where each compartment contains a partial library which contains K₀=B₀/W₀ variants,
c) Production of biomolecules in the compartments and testing of the biomolecules obtained in the single compartments for a specified phenotype, whereas from the observed phenotype no direct conclusions on the genotype can be made,
d) Selection of at least one compartment, which contains biomolecules fulfilling the wanted properties,
e) Division of the partial library contained in the selected compartment into further compartments, and
f) n-fold repetition of the steps c) to e) until in every compartment maximally only one variant (Kₙ<=1) of the gene sequence coding for the biomolecule is contained.

2. The method of claim 1, wherein the wanted property is a biocatalytic activity.

3. The method of claim 1, wherein in step c) also an amplification of the partial library takes place in the compartments up to an number of individuals V₀(x) at the point in time×per compartment, whereas the number of individuals V₀(x) divided by the number of clones per compartment K₀ gives the amplification factor F₀(x) per clone.

4. The method of claim 1, wherein in step e) the division is carried out under dilution of the partial library by means of factor F₀(x), so that in a given volume every clone contained in the compartment is statistically present up to a number X₀<W₁, this volume is divided up in a number of new compartments W₁, whereas the new number of clones per compartment amounts to K₁=X₀*K₀/W₁.

5. The method of claim 1, wherein the variant library contains 103 to 10¹⁵ variants of the gene sequence of the biomolecule.

6. The method of claim 1, wherein in step b) the variant library is divided up in 10¹ to 10⁴ compartments.

7. The method of claim 1, wherein in step b) the variant library is transferred into an organism before division.

8. The method of claim 7, wherein in step c) the culture of the organism after division is amplified to a number of organisms of 10⁸ to 10⁹ per compartment.

9. The method of claim 7, wherein the organisms also conduct the production of the biomolecules.

10. The method of claim 7, wherein the partial libraries in the compartments are re-isolated from the organisms, and the production of the biomolecules is conducted by cell-free systems.

11. The method of claim 1, wherein the amplification of the partial libraries and the production of the biomolecules is conducted by cell-free systems.

12. The method of claim 1, wherein the variant library consists of DNA-plasmids, which contain the gene sequence coding for the biomolecule.

13. The method of claim 1, wherein the variant library consists of linear nucleic acid molecules, which contain the gene sequence coding for the biomolecule.

14. The method of claim 1, wherein the biomolecules are enzymes or ribozymes or other biomolecules, which exhibit a biocatalytic activity.

15. The method of claim 1, wherein the test for a biocatalytic activity is conducted with a physical detection, method selected from the group consisting of UVIVIS-spectroscopy, fluorescence spectroscopy and fluorescencecorrelation-spectroscopy.
